(19) United States
(12) Patent Application Publication
(10) Pub. No.: US 2003/0024536 A1
(43) Pub. Date: Feb. 6, 2003

(54) Anatomical device

(76) Inventor: Richard P. Bagby, Paso Robles, CA (US)

Correspondence Address: James M. Duncan, Klein DeNatale Goldner Cooper Rosenlieb & Kimball, P O Box 11172, Bakersfield, CA, 93389-1172, US

(21) Appl. No.: 09/922,188

(22) Filed: Aug. 3, 2001

(51) Int. Cl. A61F 5/37

(52) U.S. Cl. 128/883

(57) ABSTRACT

An anatomical device for attachment to a penis is disclosed, having a frame wherein the members of the frame define either an elliptical or triangular opening for receiving the penis. The frame has a locked position and an unlocked position. Locking means are providing for engaging the device in the locked position. The frame may either be of a single piece flexible construction, or have adjacent members attached with pivoting attachment means. Use of the device applies pressure to the dorsal vein of the penis thereby restricting blood flow out of the penis, but the device applies limited pressure to the urethra.

### BACKGROUND OF THE INVENTION

The present invention relates to an anatomical device for attachment to a penis for applying local pressure to the dorsal vein of the penis and restricting the flow of blood out of the penis. It has long been known that the restriction of the superficial dorsal vein of the male reproductive organ, or penis, to maintain an erection has therapeutic value in physically and psychologically impotent males. However, restriction of the urethra is to be avoided if at all possible.

Numerous devices have been proposed for enabling a human male to achieve and maintain an erection, including surgical implants, vacuum devices, various sleeves and cylinders, and constriction devices. Among these devices are a type which partially or fully encircle the base of the penis in such a manner as to apply pressure to the dorsal vein to restrict blood flow. The present inventor previously disclosed in U.S. Pat. No. 3,794,020 an anatomical device which employed a rigid frame in the form of a U-shaped member to accomplish such blood flow restriction. U.S. Pat. No. 4,203,432 (Koch) discloses a split ring of flexible but relatively inelastic material in which the ends of the ring are secured together with an elastic band. U.S. Pat. No. 5,306,227 discloses an elastic ring having a predetermined dorsal ring area for engaging the dorsal vein and a U-shaped channel to aligned with the urethra so as to allow unrestricted flow. U.S. Pat. No. 5,327,910 discloses a device having a closed loop which has rigid portions interconnected by a deformable portion, the rigid portions to constrict the dorsal vein and urethra. U.S. Pat. No. 5,421,324 (Kelly) discloses a truss comprising a linear rigid member which is pulled into contact with the dorsal vein by a an elastic band which passes around the circumference of the penis. The truss disclosed in U.S. Pat. No. 5,526,803 (Kelly), purporting to be an improvement over the device disclosed in the '324 patent, comprises an elongated cylindrical member, divided along its longitudinal axis, through which a loop passes through. The two ends of the loop are fastened on the underside of the penis. The device disclosed in U.S. Pat. No. 5,695,444 (Chaney) is an elastic constrictor ring having a radially extending protuberance for bearing against the dorsal vein. U.S. Pat. No. 5,810,710 (Burgos) discloses a split ring having releaseable fastening means. In U.S. Pat. No. 5,873,813 (Weiss), a resilient ring member having a circular spring is disclosed, where the inside circumferential surface of the ring member contracts the penis. U.S. Pat. No. 5,954,631 (Gorsuch) discloses a band which is wrapped around the penis. U.S. Pat. No. 5,997,469 (Northcutt) discloses a ring or set of rings which encircles the base of the penis.

Almost all of the aforementioned devices have circular openings for engaging the penis, many which fully encircle the organ. However, in cross-section, the male penis is actually more elliptical than circular. In addition, many of the disclosed devices apply pressure to the urethra, or fail to provide the optimal pressure to the dorsal vein. Finally, many of these devices are difficult to apply, often requiring elaborate manipulation at time when the user is often preoccupied with other concerns. An anatomical device is needed which provides the optimal pressure to the dorsal vein, does not restrict flow from the urethra, and which is simple to install.

### SUMMARY OF THE INVENTION

The present invention is directed to an anatomical device which meets the needs identified above.

The disclosed apparatus is a an anatomical device adapted for attachment to a penis, comprising a flexible frame having a top, a left leg and a right leg, the left leg and right leg having distal ends, the left leg having an left engaging section, the right leg having a right engaging section, the left engaging section and right engaging section in spaced-apart facing relation, the top, left engaging section and right engaging section defining an elliptical opening for receiving the penis. The left leg engaging section has a lower left engaging portion and the right engaging section has a lower right engaging portion, the lower left engaging portion and lower right engaging portion are each configured to engage the underside of the penis. The flexible frame has an unlocked position, wherein the size of the elliptical opening defined by the top, left engaging section and right engaging section may manually be adjusted and a locked position in which the size of said elliptical opening may not manually be adjusted. Finally, means are provided for engaging the flexible frame in the locked position.

An alternative embodiment, rather than using a flexible frame, utilizes pivoting means connecting the left leg and the right leg. In this embodiment, the frame for attachment to the penis has a left leg and a right leg, the left leg and right leg each having proximal and distal ends, the proximal end of the left leg attached to the proximal end of the right leg with pivoting attachment means. The left leg has a left engaging section and the right leg has a right engaging section, the left engaging section and right engaging section being in spaced-apart facing relation. The left engaging section and right engaging section define an elliptical opening for receiving the penis. The left leg engaging section has a lower left engaging portion and the right engaging section has a lower right engaging portion, the lower left engaging portion and lower right engaging portion each configured to engage the underside of the penis. The frame has an unlocked position, in which the size of the elliptical opening defined by the left engaging section and right engaging section may manually be adjusted.

The frame also has a locked position in which the size of the elliptical opening is not manually adjustable. Means are provided for engaging the frame in the locked position.

It has been found that the best shape of ellipse for the disclosed device is an ellipse of 45 degrees to 50 degrees. An embodiment of the present device having this shape is disclosed. It has also been found that there is an optimum range of angles for cradling or supporting the underside of the penis, namely 25 degrees to 35 degrees. At this range of angles, there is a dramatic increase in the support of the penis from its lower side, causing it to be pushed upwards more efficiently against the upper engaging sections of the device, thus exerting greater localized pressure against the superficial dorsal vein.

Another embodiment of the anatomical device is disclosed having many of the same features described above, but in which a triangular opening is defined as opposed to an eliptical opening. The triangular opening may be fashioned in such a manner as to provide many of the same benefits as the elliptical opening. In one variation of this embodiment, the lower left engaging portion and lower right engaging portion respectively engage the underside of the penis at an angle from 40 degrees to 45 degrees, which results in more local pressure against the superficial dorsal vein.

These and other features, aspects, and advantages of the present invention will become better understood with regard to the following description, appended claims, and accompanying drawings.

### BRIEF DESCRIPTION OF THE DRAWINGS

FIG. 1 is a front view of the flexible frame embodiment of the disclosed device, showing the device in the locked position using one means of locking the device.

FIG. 2 is a front view of the flexible frame embodiment of the disclosed device, showing the device in the unlocked position.

FIG. 3A shows a sectional view of the round embodiment of the flexible frame.

FIG. 3B shows a sectional view of the rectangular embodiment of the flexible frame.

FIG. 4 shows a perspective view of one hinged embodiment of the disclosed device.

FIG. 5 shows a top view of the hinged embodiment of FIG. 4.

FIG. 6 shows a bottom view of the hinged embodiment of FIG. 4.

FIG. 7 shows a front view of the hinged embodiment of FIG. 4 in the locked position.

FIG. 8 shows a sectional view of the hinged embodiment of FIG. 4.

FIG. 9 shows the hinged embodiment of FIG. 4 in the unlocked position.

FIG. 10 shows a front view of a hinged embodiment of the disclosed device in the locked position.

FIG. 11 shows a front view of the hinged embodiment of FIG. 10 in the unlocked position.

FIG. 12 shows a sectional view of the hinged embodiment of FIG. 10.

FIG. 13 is a front view of a hinged embodiment of the disclosed device, where a collar is used as a means of maintaining the device in the locked position.

FIG. 14 shows a top view of the collar shown in FIG. 15.

FIG. 15 shows a perspective view of an alternative embodiment of the disclosed device having a tongue and groove hinge mechanism.

FIG. 16 is a top view of the tongue and groove mechanism shown in FIG. 13.

FIG. 17 shows a front view of a hinged embodiment in the locked position.

FIG. 18 shows a front view of a hinged embodiment in the unlocked position.

FIG. 19. shows a front view of a hinged embodiment in the locked position.

FIG. 20 shows a front view of a hinged embodiment in the unlocked position.

FIG. 21 shows a front view of a hinged embodiment in the locked position.

FIG. 22 shows a front view of a hinged embodiment in the unlocked position.

FIG. 23 shows a front detailed view of a locking mechanism in the locked position.

FIG. 24 shows a front detailed view of a locking mechanism in the unlocked position.

FIG. 25 shows an embodiment in the locked position where the ends of a spring have been fastened to the right leg and left leg of the embodiment.

FIG. 26. shows the embodiment of FIG. 25 in the unlocked position where the ends of a spring have been fastened to the right leg and left leg of the embodiment.

FIG. 27. shows a front view of a hinged embodiment in the locked position.

FIG. 28 shows a front view of a hinged embodiment in unlocked position.

FIG. 29 shows a front view of a hinged embodiment in the locked position.

FIG. 30 shows a front view of a hinged embodiment in the unlocked position.

FIG. 31 shows a top view of the hinged embodiment of FIG. 29.

FIG. 32 shows a front view of a hinged embodiment in the locked position.

FIG. 33 shows a front view of a hinged embodiment in the unlocked position.

FIG. 34 shows a detailed view of the hinge of the hinged embodiment of FIG. 32.

FIG. 35 shows a front view of a flexible frame embodiment of the disclosed device.

FIG. 36 shows a sectional view of the locking mechanism of the embodiment of FIG. 35

FIG. 37 shows a front view of a hinged embodiment in the locked position.

FIG. 38 shows a front view of a hinged embodiment in the unlocked position.

FIG. 39. shows a perspective view of a flexible frame embodiment of the disclosed device, the flexible frame having a triangular opening in the locked position.

FIG. 40 shows a front view of the embodiment of FIG. 39 in the unlocked position.

FIG. 41 shows a front view of a hinged embodiment in the locked position.

FIG. 42. shows a front view of a hinged embodiment in the unlocked position.

FIG. 43. shows a perspective view of a hinged embodiment in the locked position.

FIG. 44 shows a front view of a hinged embodiment in the unlocked position.

FIG. 45 shows a front view of a hinged embodiment in the locked position.

FIG. 46 shows a front view of a hinged embodiment in the unlocked position.

FIG. 47 shows a sectional view of the hinge mechanism of the embodiment shown in FIG. 45.

FIG. 48 shows a front view of a hinged embodiment in the locked position.

FIG. 49 shows a front view of a hinged embodiment in the unlocked position.

FIG. 50 shows a perspective view of a flexible frame embodiment of the disclosed device having an elliptical opening wherein a latch is used as a locking means.

FIG. 51 shows a front view of the embodiment of FIG. 50 with the latch in place.

FIG. 52 shows a front view of the embodiment of FIG. 50 with the latch removed.

FIG. 53 shows a sectional view of the embodiment shown in FIG. 50.

FIG. 54 shows a perspective view of a flexible frame embodiment of the disclosed device having a triangular opening wherein a latch is used as a locking means.

FIG. 55 shows a front view of the embodiment of FIG. 54 with the latch in place.

FIG. 56 shows a front view of the embodiment of FIG. 54 with the latch removed.

FIG. 57 shows a sectional view of the embodiment shown in FIG. 54.

### DETAILED DESCRIPTION OF THE EMBODIMENTS

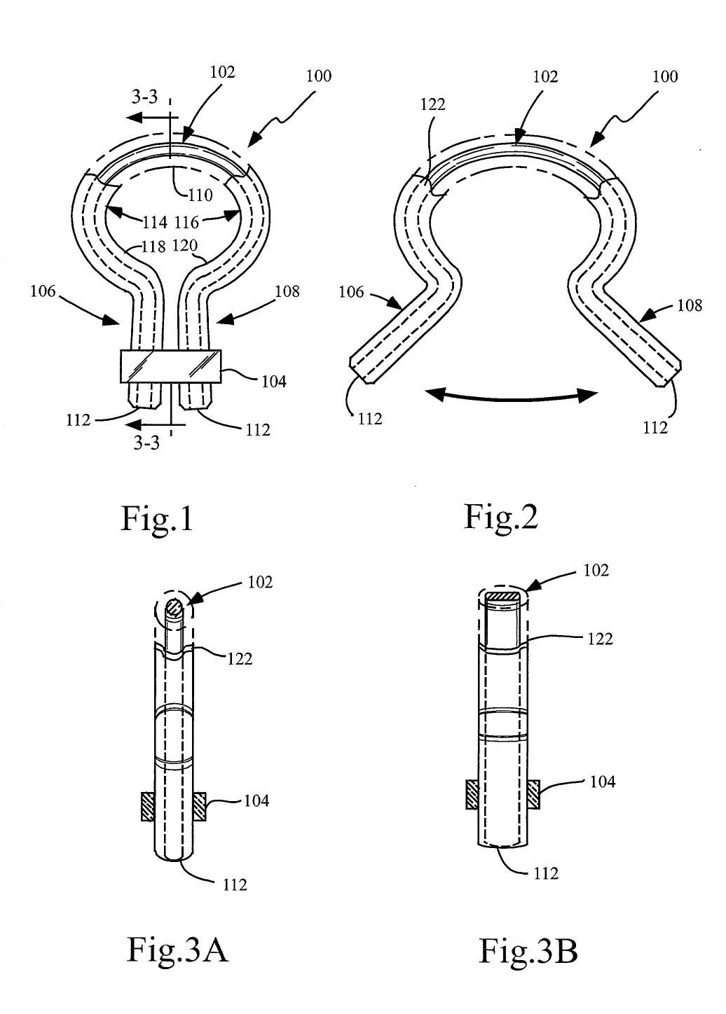

Referring now specifically to the drawings, FIG. 1 through FIG. 4 show a flexible frame embodiment 100 of the present device. The major components of this embodiment are the flexible frame 102 and a latex loop 104 which acts as locking means for retaining the device in the locked position. The flexible frame is comprised of the left leg 106, the right leg 108 and the top 110. The left leg 106 and right leg 108 have distal ends 112. The left leg 106 has left engaging section 114 and the right leg 108 has right engaging section 116. The adjacent curved surfaces of the top 110, the left engaging section 114 and the right engaging section 116 define an elliptical opening which engages the penis. In one embodiment, the elliptical opening forms a 45 degree to 50 degree ellipse. It has been found that an elliptical opening of 45 degrees to 50 degrees provides optimal support for the penis. Left engaging section 114 and right engaging section 116 have lower portions, respectively the lower left engaging portion 118 and the lower right engaging portion 120. In one embodiment, the lower left engaging portion 118 and the lower right engaging portion 120 form a cradle for the underside of the penis, the lower left engaging portion 118 and the lower right engaging portion 120 each engaging the organ at an angle ranging from 25 degrees to 35 degrees. It has been found that an angle of approximately 30 degrees dramatically increases the support of the penis from its lower side, causing it to be pushed upwards more efficiently against the top 110 of the device, thus exerting greater localized pressure against the superficial dorsal vein and restricting the flow of blood out of the penis.

The flexible frame 102 may be constructed of a flexible plastic such as Lexan, PVC or similar plastic or other material having similar properties. As shown in FIG. 2, in the unlocked position the left leg 106 and right leg 108 can easily be spread widely apart for easy application of the device 100 to an erect penis. After the device 100 is applied to the penis, the device is placed in the locked position by slipping latex loop 104 over the distal ends 112 of the left leg 106 and right leg 108. The flexible frame 102 may have a round cross-section, as shown in FIG. 3A, or a rectangular cross-section, as shown in FIG. 3B. Both cross-sections provide sufficient rigidity to maintain their elliptical shape to fit the natural elliptical contour of the penis, while providing complete flexibility for easy application or removal. The round cross-section makes a slim width of ⅜ inch possible, which the rectangular cross-section allows for a minimal width of approximately ½ inch. The flexible frame 102 is completely covered with a thick, soft latex skin 122, which affords complete flexibility, comfort and easy sanitation.

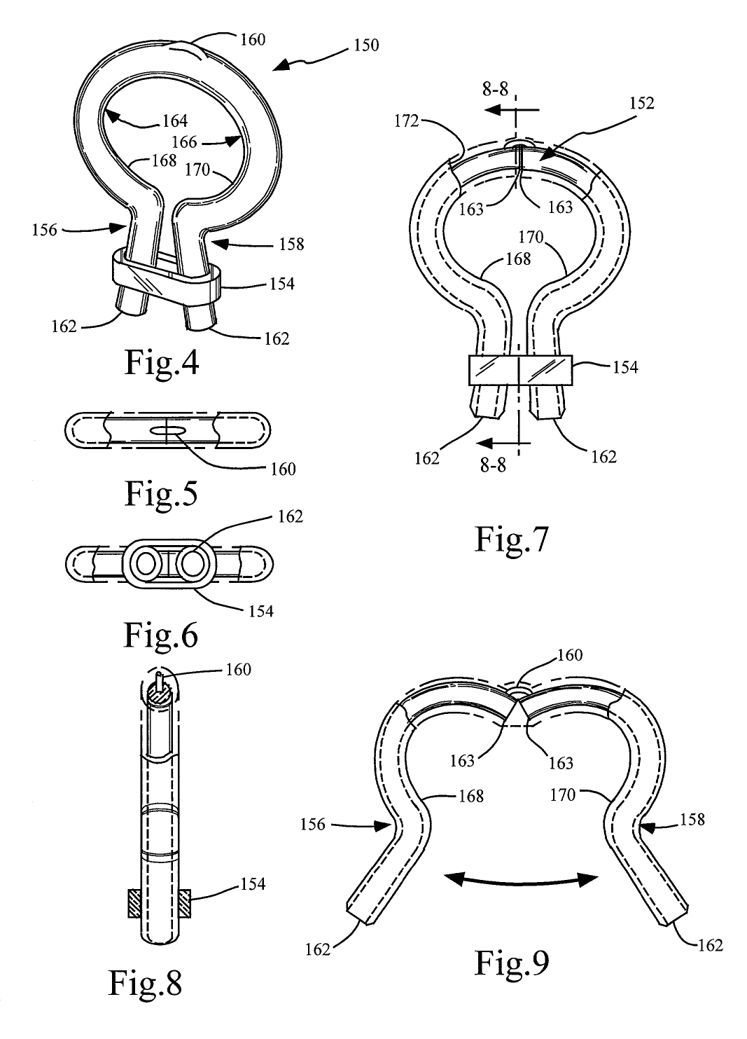

FIG. 4 through FIG. 9 show a hinged embodiment 150 of the present device. The major components of this embodiment are the frame 152 and a latex loop 154 which acts as locking means for retaining the device in the locked position. The frame is comprised of the left leg 156, the right leg 158 and the hinge 160. The left leg 156 and right leg 158 have distal ends 162 and proximal ends 163. The proximal ends 163 are joined together with pivoting attachment means, such as a hinge 160. A suitable hinge 160 is a flexible nylon strand attached to each proximal end.

The left leg 156 has left engaging section 164 and the right leg 158 has right engaging section 166. The adjacent curved surfaces of the left engaging section 164 and the right engaging section 166 define an elliptical opening which engages the penis. In one embodiment, the elliptical opening forms a 45 degree to 50 degree ellipse. It has been found that an elliptical opening of 45 degrees to 50 degrees provides optimal support for the penis. Left engaging section 164 and right engaging section 166 each have lower portions, respectively the lower left engaging portion 168 and the lower right engaging portion 170. In one embodiment, the lower left engaging portion 168 and the lower right engaging portion 170 form a cradle for the underside of the penis, the lower left engaging portion 168 and the lower right engaging portion 170 each engaging the organ at an angle ranging from 25 degrees to 35 degrees. It has been found that an angle of approximately 30 degrees dramatically increases the support of the penis from its lower side, causing it to be pushed upwards more efficiently against the curved area adjacent to the proximal ends 163, thus exerting greater localized pressure against the superficial dorsal vein and restricting the flow of blood out of the penis.

The frame 152 may be constructed of a wide variety of materials, including plastic, metal, metal alloy or composites. As shown in FIG. 9, in the unlocked position the left leg 156 and right leg 158 can easily pivot at the hinge 160 for easy application of the device 150 to an erect penis. After the device 150 is applied to the penis, the device is placed in the locked position by slipping latex loop 154 over the distal ends 162 of the left leg 156 and right leg 158. The frame 152 may have a round cross-section or a rectangular cross-section. The frame 152 is completely covered with a thick, soft latex skin 172, which affords complete flexibility, comfort and easy sanitation.

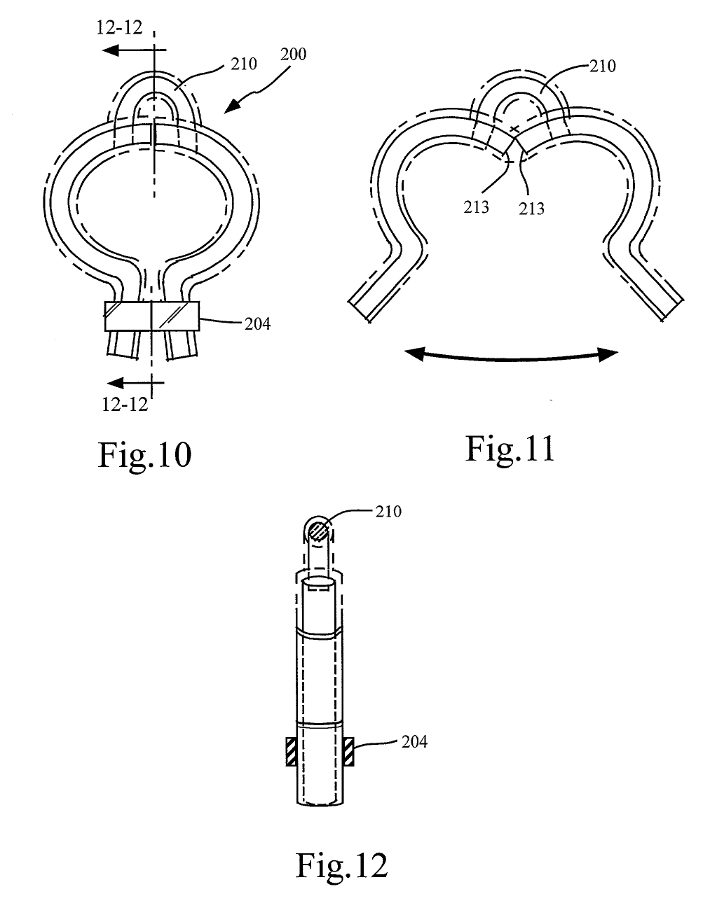

FIG. 10 through FIG. 12 show a hinged embodiment 200 of the present device. This embodiment is identical to hinged embodiment 150 except the proximal ends 213 of hinged embodiment 200 are joined together with pivoting attachment means which has a higher pivot point such as U-shaped hinge 210. Hinge 210 may be fabricated from PVC or Lexan. Proximal ends 213 may also be fabricated with a protrusion which may be arranged by the user to apply local pressure to the dorsal vein of the penis

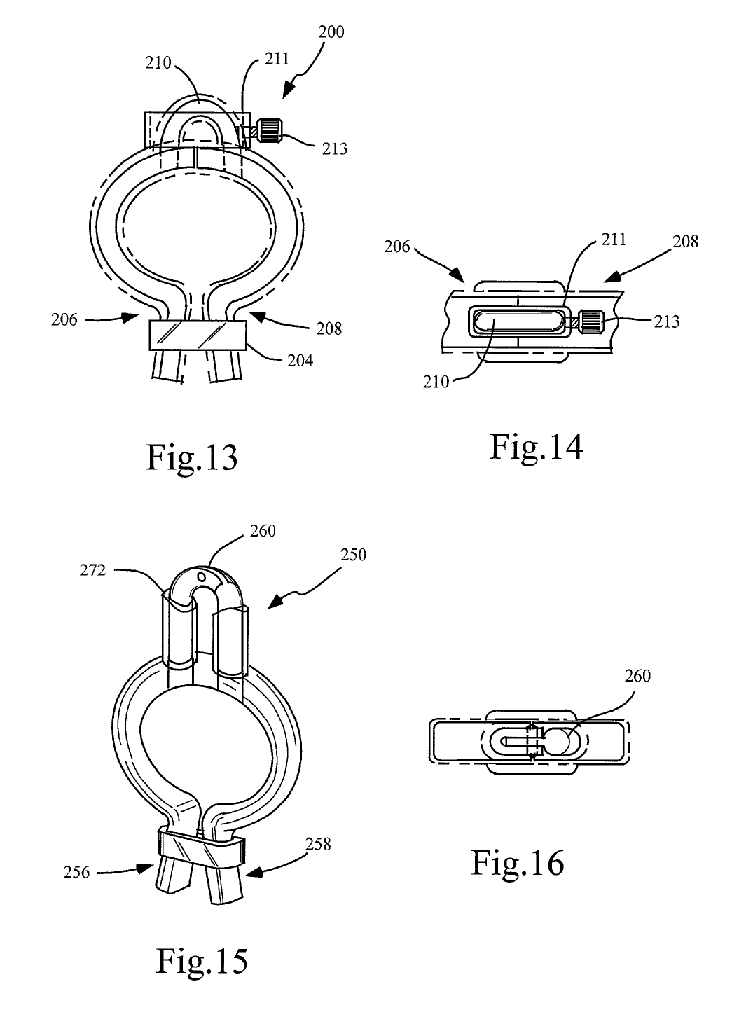

FIG. 13 and FIG. 14 show hinged embodiment 200 to which collar 211 is installed over hinge 210, to secure left leg 206 and right leg 208, such that collar 211 provides means for retaining device 200 in the locked position. Latex loop 204 is optional if collar 211 is used. If latex loop 204 is not used, complete clearance around the urethra is assured. Adjustment screw 213 enables a range of adjustments in the locked position. To reach the maximum opening, collar 211 is easily removed. Collar 211 may be made of metal with a latex skin. Hinge 210 need not have a latex skin, the absence of the skin facilitating the application or removal of collar 211.

FIG. 15 and FIG. 16 show a hinged embodiment 250 of the present device. This embodiment is identical to hinged embodiment 200 except the pivoting attachment means of this embodiment comprises a tongue and groove pivot mechanism 260 for ease of movement of left leg 256 and right leg 258. Pivot mechanism 260 is entirely covered with latex skin 272.

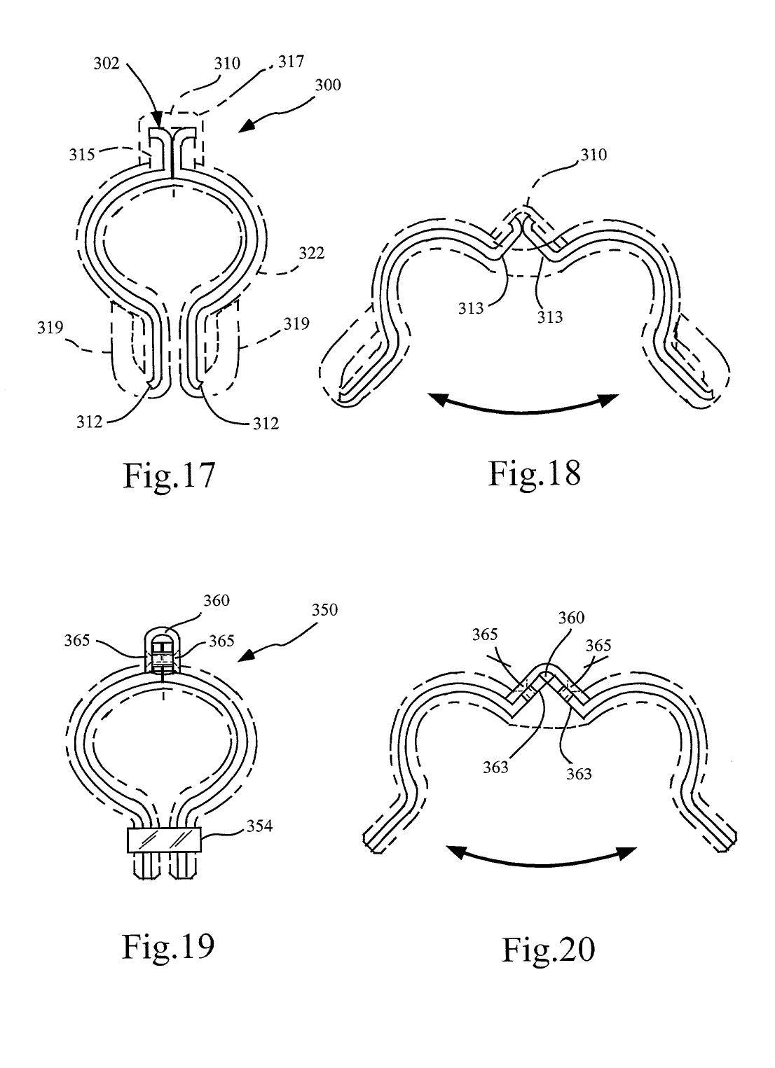

FIG. 17 and FIG. 18 show hinged embodiment 300 of the present device. In this embodiment, frame 302 is made from a rigid material such as metal or hard plastic. Proximal ends 313 converge at the top of the elliptical configuration and are attached to one another by inner elastic band 315 and outer elastic band 317, which, in conjunction provide the pivoting attachment means of this embodiment. Inner elastic band 315 and outer elastic band 317 also provide means for retaining device 300 in the locked position. Frame 302 may have either a rectangular or round cross-section. As shown in FIG. 17, latex handles 319 may be fabricated from the latex skin which may encase frame 302, such that the handles 319 extend past distal ends 312 of frame 302 and attach to the latex skin 322. Handles 319 facilitate spreading the device to apply it or remove it. Device 300 maintains sufficient tension to accommodate various sized erections while not interfering with ejaculation. Device 300 may be applied or removed at any time, before or during intercourse.

FIG. 19 and FIG. 20 show hinged embodiment 350 of the present device. In this embodiment, proximal ends 363 are connected to nylon hinge 360 which acts as the pivoting attachment means. Proximal ends 363 are attached to nylon hinge 360 with screws 365. Proximal ends 363 are threaded to receive screws 365. Latex band 354 provides means for retaining device 350 in the locked position.

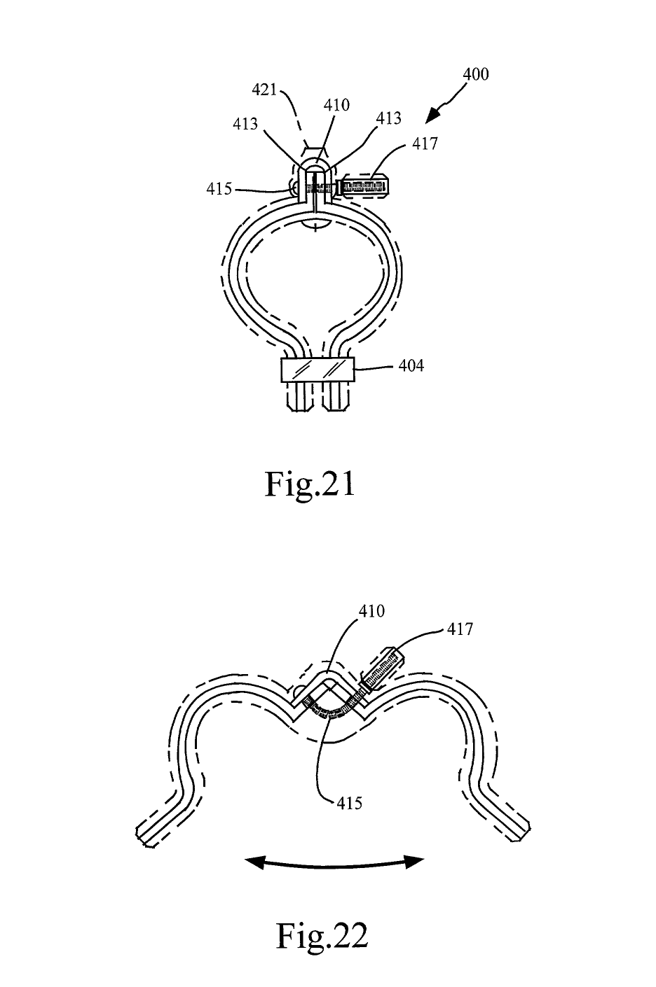

FIG. 21 and FIG. 22 show hinged embodiment 400 of the present device. In this embodiment, proximal ends 413 are connected to nylon hinge 410 which acts as the pivoting attachment means. Proximal ends 413 are drilled to receive flexible screw 415, which may be made of nylon. Nut 417 is made up on flexible screw 415 to provide means for engaging and retaining device 400 in the locked position. A latex collar 421 may be slipped over hinge 410 and proximal ends 413 to provide additional means of retaining the device in the locked position. Use of latex band 404 as means for retaining device 400 in the locked position is optional.

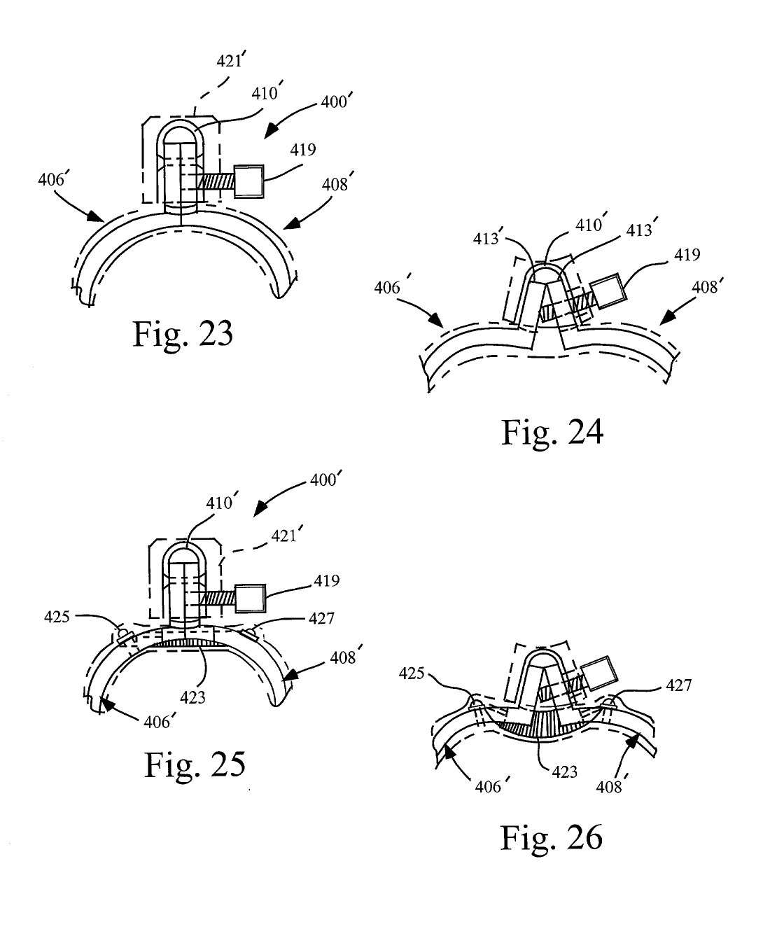

FIG. 23 and FIG. 24 show alternative means for retaining hinged embodiment 400′ device in the locked position and for placing the device in the unlocked position. Proximal ends 413′ are connected to nylon hinge 410′ which acts as the pivoting attachment means. One of proximal ends 413′ is drilled through and threaded. A latex collar 421′ is slipped over hinge 410′ and proximal ends 413′, fitting sufficiently tight to retain proximal ends 413′ abutted together and retaining the device in the locked position. Screw 419′ is inserted through the threaded hole in one of the proximal ends and adjusted to abut the adjacent proximal end 413′. The device 400′ is disengaged from the locked position by tightening screw 419′ so as to displace the proximal end 413′ of the left leg 406′ from the proximal end 413′ of the right leg 408′.

As shown in FIG. 25, in addition to or instead of using latex collar 421′ as means of retaining device 400′ in the locked position, a round spring 423, having left end 425 and right end 427 may be used. Left end 425 and right end 427 are flared or have eyes, and have a larger diameter than the body of spring 423. A hole of larger diameter than spring 423 but of smaller diameter than left end 425 is placed in left leg 406′. Likewise, a hole of larger diameter than spring 423 but of smaller diameter than right end 427 is placed in right leg 408′.

As shown in FIG. 25 and FIG. 26, a portion of spring 423 projects downward, thus providing additional localized pressure on the superficial dorsal vein. An additional advantage of spring 423 is that it bridges the gap between left leg 406′ and right leg 408′, so there will always be a continuous, smooth, round projection pressing against the top side of the penis, regardless of the relative positions of the left leg 406′ and right leg 408′. The spring 423 be covered with a latex skin for added comfort.

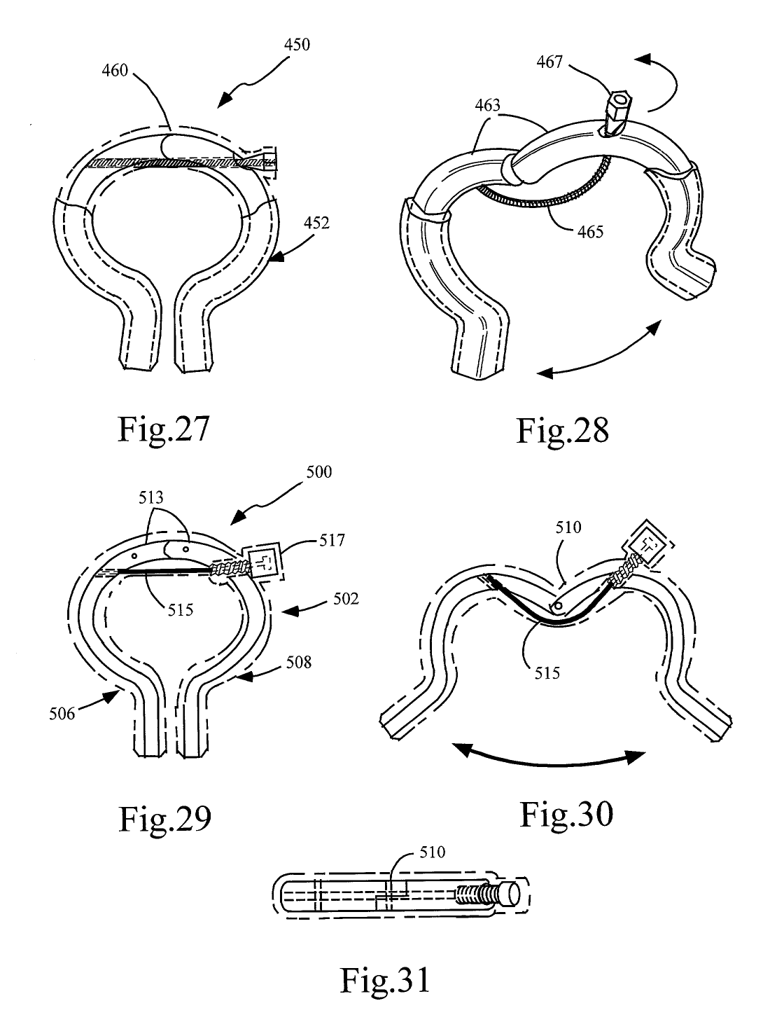

FIG. 27 and FIG. 28 show hinged embodiment 450 of the present device. The frame 452 is made of a sturdy plastic. In this embodiment, proximal ends 463 are pivotally connected with hinge 460, which may be a tongue and groove hinge, a pin-type hinge, or other hinge known in the art. Proximal ends 463 are drilled to receive flexible screw 465, which may be made of nylon. Nut 467 is made up on flexible screw 465 to provide means for engaging and retaining device 450 in the locked position.

FIG. 29 through FIG. 30 show hinged embodiment 500 of the present device. The frame 502 is made of either sturdy plastic or metal. In this embodiment, proximal ends 513 are pivotally connected with hinge 510, which may be a tongue and groove hinge, a pin-type hinge, or other hinge known in the art. Proximal ends 513 are drilled to receive cable 515 and screw 517. Screw 517 is metal and employs left-handed acme threads. Cable 515 is secured on one proximal end 513 and the opposing proximal end 513 is drilled with a left-handed thread to receive screw 517. As screw 517 is turned clockwise, cable 515 is drawn tight and pulls left leg 506 and right leg 508 toward each other. FIG. 31 shows a top view of the hinge 510 and screw 517 of device 500.

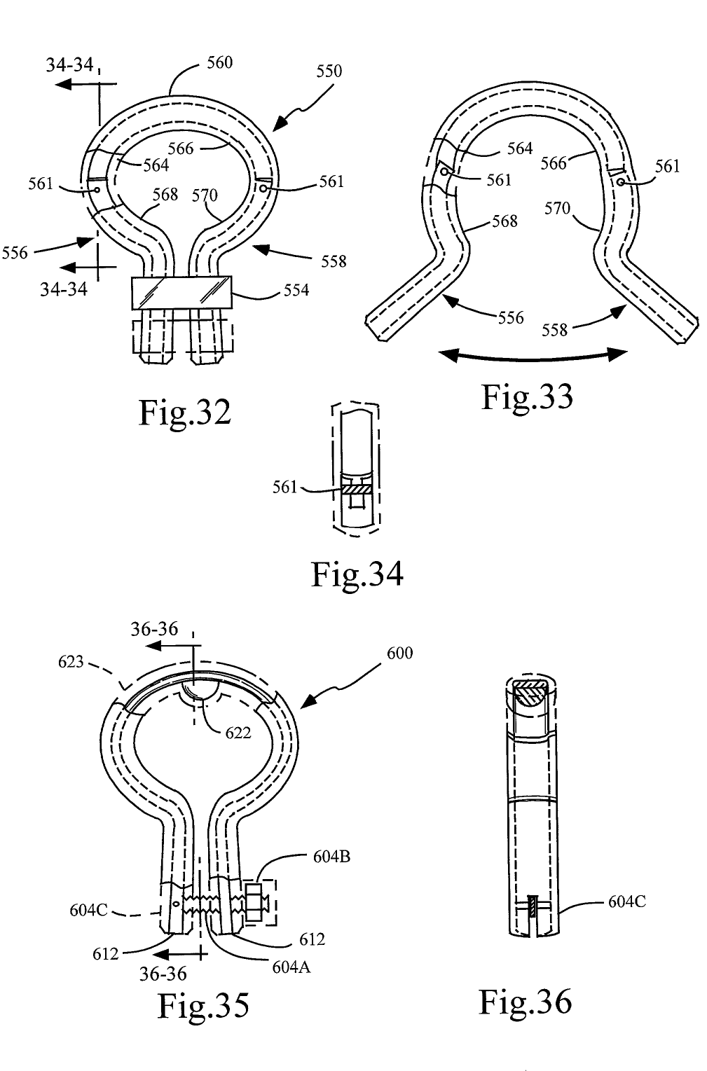

FIG. 32 through FIG. 34 show hinged embodiment 550 of the present device. This embodiment has a top 560, left leg 556 and right leg 558. Left leg 556 is comprised of upper left engaging section 564 and lower left engaging section 568. Likewise, right leg 558 is comprised of upper right engaging section 566 and lower right engaging section 570. In this embodiment, a separate hinge 561 is used for left leg 556 and right leg 558. Upper left engaging section 564 is pivotally attached to lower left engaging section 568 and upper right engaging section 566 is pivotally attached to lower right engaging section 570. As shown in FIG. 34, hinge 561 is a tongue and groove with penis. Latex loop 554 acts as a locking means for retaining the device in the locked position.

FIG. 35 and FIG. 36 show a flexible frame embodiment 600 of the present device. In this embodiment, the locking means are provided by a screw 604A, nut 604B and screw pivot 604C. Screw 604A, flattened on one end, is pivotally attached within a slot in distal end 612 by screw pivot 604C. As nut 604B is tightened on screw 604A, the distal ends 612 are drawn together. A projection 623 may be molded into rubber skin 622 for additional localized pressure on the superficial dorsal vein.

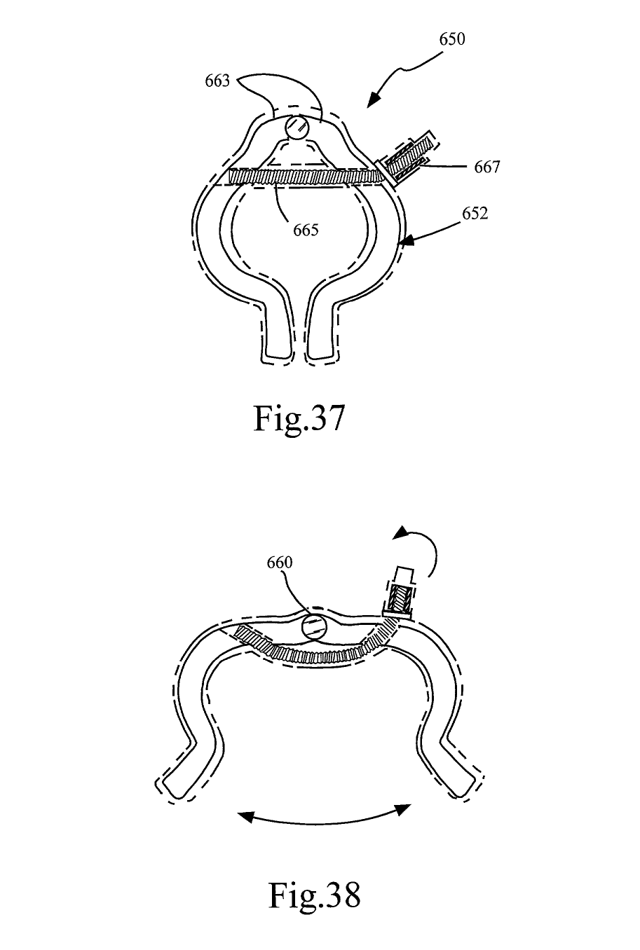

FIG. 37 and FIG. 38 show hinged embodiment 650 of the present device. The frame 652 is made of a sturdy plastic or metal. In this embodiment, proximal ends 663 are pivotally connected with hinge 660, which is a pin-type hinge. Proximal ends 663 are drilled to receive flexible screw 665, which may be made of nylon. Nut 667 is made up on flexible screw 665 to provide means for engaging and retaining device 650 in the locked position.

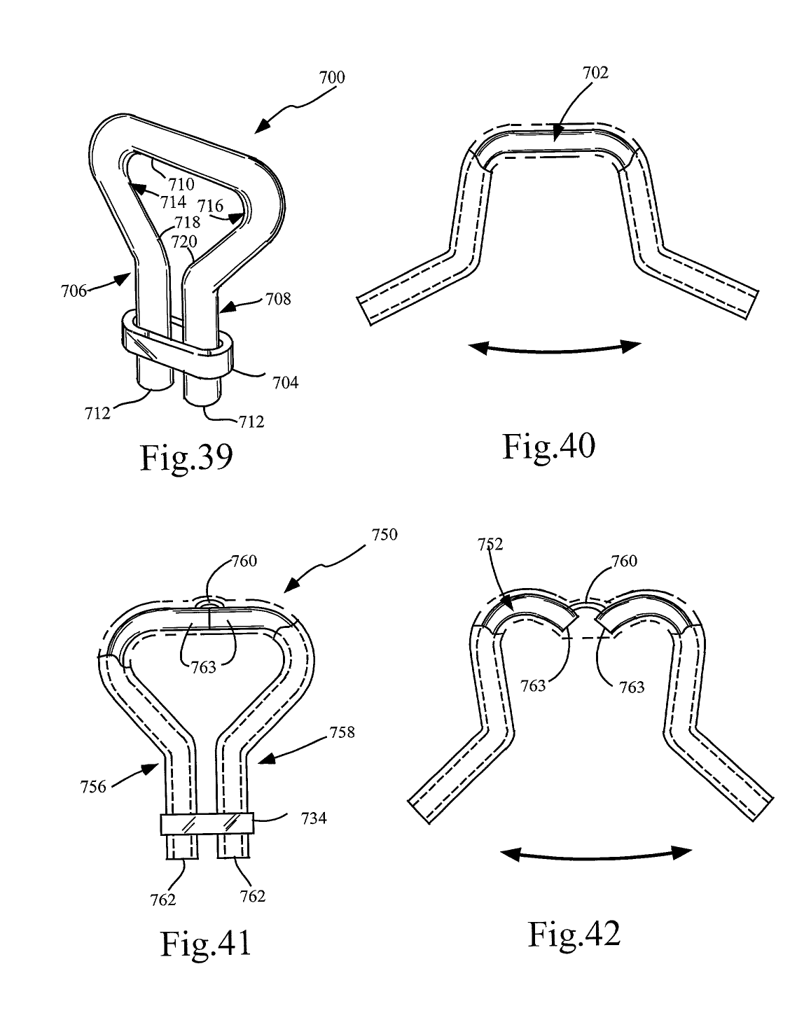

FIG. 39 and FIG. 40 show flexible frame embodiment 700 of the present device. The major components of this embodiment are the flexible frame 702 and a latex loop 704 which acts as locking means for retaining the device in the locked position. The flexible frame is comprised of the left leg 706, the right leg 708 and the top 710. The left leg 706 and right leg 708 have distal ends 712. The left leg 706 has le ft engaging section 714 and the right leg 708 has right engaging section 716. The adjacent surfaces of the top 710, the left engaging section 714 and the right engaging section 716 define a triangular opening which engages the penis. Left engaging section 714 and right engaging section 716 each have lower portions, respectively the lower left engaging portion 718 and the lower right engaging portion 720. In this embodiment, the lower left engaging portion 718 and the lower right engaging portion 720 form a cradle for the underside of the penis, the lower left engaging portion 718 and the lower right engaging portion 720 each engaging the organ at an angle ranging from 40 degrees to 45 degrees.

FIG. 41 and FIG. 42 show a hinged embodiment 750 of the present device having a triangular opening. The major components of this embodiment are the frame 752 and a latex loop 754 which acts as locking means for retaining the device in the locked position. In this embodiment, frame 752 is made from a rigid material such as metal or hard plastic. The frame is comprised of the left leg 756, the right leg 758 and the hinge 760. The left leg 756 and right leg 758 have distal ends 762 and proximal ends 763. The proximal ends 763 are joined together with pivoting attachment means, such as a hinge 760. A suitable hinge 760 is a flexible nylon strand attached to each proximal end.

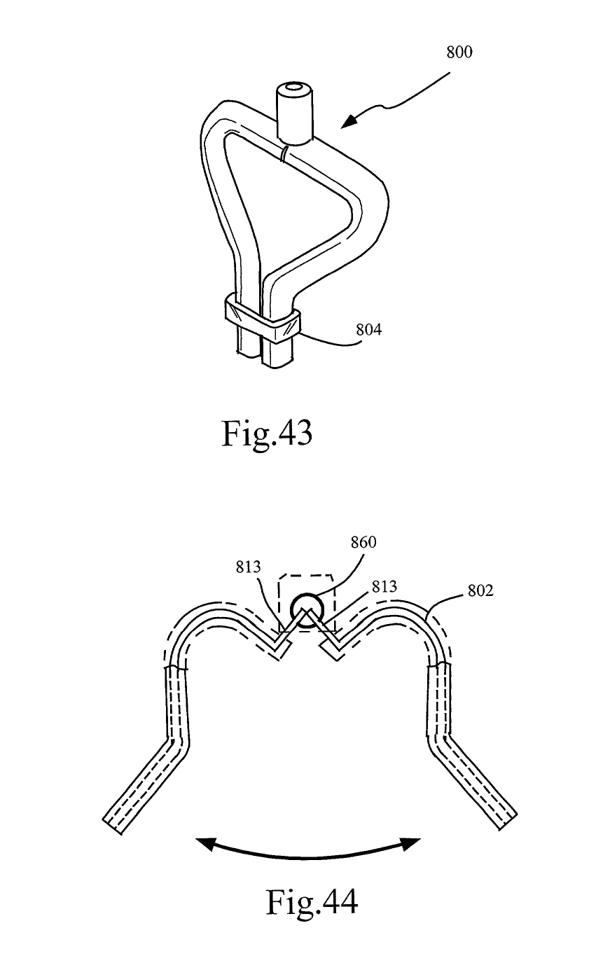

FIG. 43 and FIG. 44 show hinged embodiment 800 of the present device having a triangular opening. In this embodiment, frame 802 is made from a rigid material such as metal or hard plastic. In this embodiment, proximal ends 813 are connected to nylon hinge 860 which acts as the pivoting attachment means. Latex band 804 provides means for retaining device 800 in the locked position.

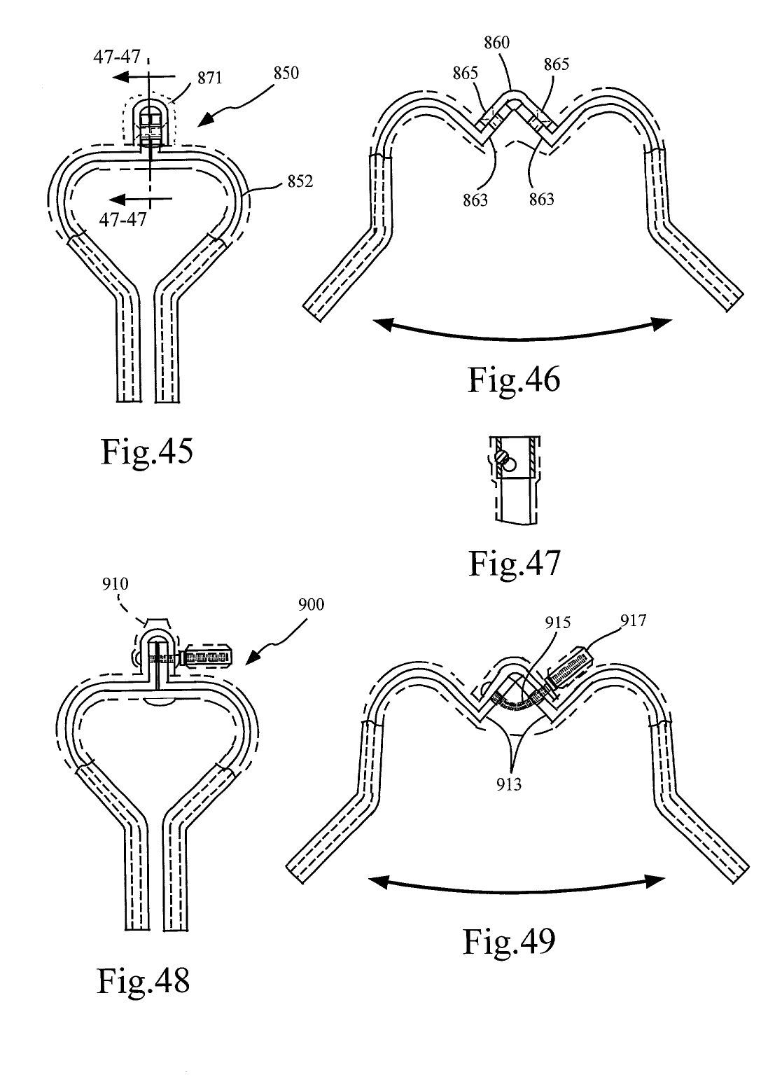

FIG. 45 through FIG. 47 show hinged embodiment 850 of the present device having a triangular opening. In this embodiment, frame 852 is made from a rigid material such as metal or hard plastic. In this embodiment, proximal ends 863 are connected to nylon hinge 860 which acts as the pivoting attachment means. Proximal ends 863 are attached to nylon hinge 860 with screws 865. Proximal ends 863 are threaded to receive screws 865. A latex collar 871 is slipped over hinge 860 and proximal ends 863, fitting sufficiently tight to retain proximal ends 863 abutted together and retaining the device in the locked position.

FIG. 48 and FIG. 49 show hinged embodiment 900 of the present device having a triangular opening. In this embodiment, proximal ends 913 are connected to nylon hinge 910 which acts as the pivoting attachment means. Proximal ends 913 are drilled to receive flexible screw 915, which may be made of nylon. Nut 917 is made up on flexible screw 915 to provide means for engaging and retaining device 900 in the locked position.

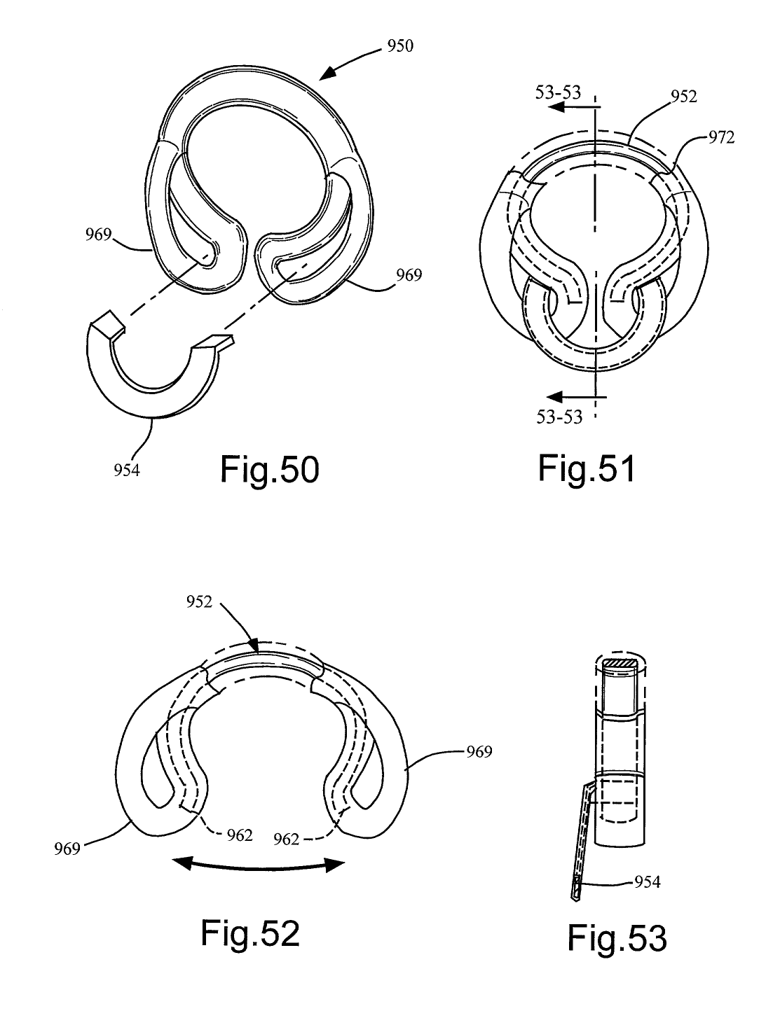

FIG. 50 through FIG. 53 show a flexible frame embodiment 950 of the present device having an elliptical opening. The major components of this embodiment are the flexible frame 952 and a rigid latch 954 which acts as locking means for retaining the device in the locked position. As shown in FIG. 51 and FIG. 52, latex handles 969 may be fabricated from the latex skin which encases frame 952, such that the handles 969 extend past distal ends 962 of frame 952 and attach to the latex skin 972. Handles 969 facilitate spreading the device to apply it or remove it. The flexible frame 952 may be constructed of a flexible plastic such as polycarbonate (Lexan), PVC or similar plastic or other material having similar properties.

Latch 954 is semi-circular and formed of a rigid material. Latch 954 may be totally encapsulated in soft rubber and fits snugly into handles 969. A tab on each side of latch 954 fits into the opening formed by a corresponding handle 969. The latch 954 provides clearance for the urethra as it curves around it in a semi-circular configuration. Latch 954 may either complete detach from the rest of the device, or one side of the latch 954 may be attached to latex skin 972 by bonding means such as glue.

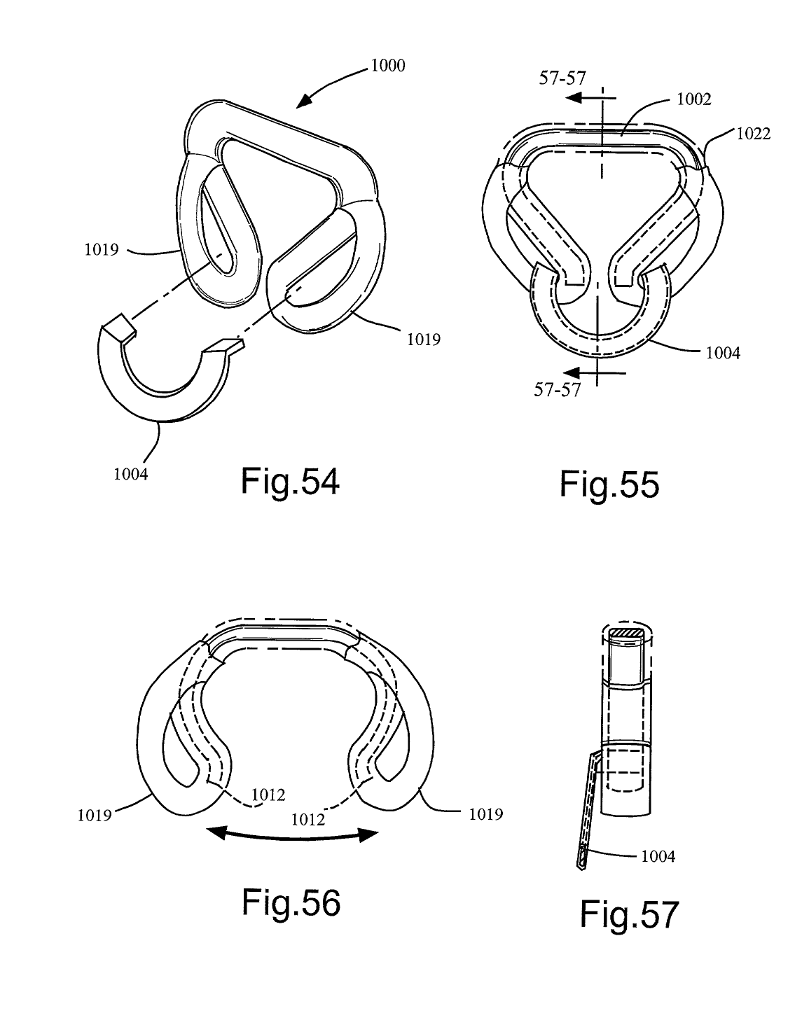

FIG. 54 through FIG. 57 show a flexible frame embodiment 1000 of the present device having an triangular opening. The major components of this embodiment are the flexible frame 1002 and a rigid latch 1004 which acts as locking means for retaining the device in the locked position. As shown in FIG. 55 and FIG. 56, latex handles 1019 may be fabricated from the latex skin which may encase frame 1002, such that the handles 1019 extend past distal ends 1012 of frame 1002 and attach to the latex skin 1022. Handles 1019 facilitate spreading the device to apply it or remove it. The flexible frame 1002 may be constructed of a flexible plastic such as polycarbonate (Lexan), PVC or similar plastic or other material having similar properties.

Latch 1004 is semi-circular and formed of a rigid material. Latch 1004 may be totally encapsulated in soft rubber and fits snugly into handles 1019. A tab on each side of latch 1004 fits into the opening formed by a corresponding handle 1019. The latch 1004 provides clearance for the urethra as it curves around it in a semi-circular configuration. Latch 1004 may either complete detach from the rest of the device, or one side of the latch 1004 may be attached to latex skin 1022 by bonding means such as glue.

While the above is a description of various embodiments of the present invention, further modifications may be employed without departing from the spirit and scope of the present invention. For example, the size, shape, and/or material of the various components may be changed as desired. Thus the scope of the invention should not be limited by the specific structures disclosed. Instead the true scope of the invention should be determined by the following claims.

What is claimed is:

1. An anatomical device adapted for attachment to a penis, comprising:
(a) a flexible frame for attachment to a penis, said flexible frame having a top, a left leg and a right leg, the left leg and right leg having distal ends, the left leg having an left engaging section, the right leg having a right engaging section, the left engaging section and right engaging section in spaced-apart facing relation, said top, left engaging section and right engaging section defining an elliptical opening for receiving the penis;
(b) said flexible frame having a unlocked position, wherein the size of the elliptical opening defined by the top, left engaging section and right engaging section may manually be adjusted and a locked position wherein the size of said elliptical opening may not manually be adjusted; and
(c) means for engaging said flexible frame in the locked position.

2. The anatomical device of claim 1 wherein the elliptical opening defined by the top, left engaging section and right engaging section in the locked position is a 45 degree to 50 degree ellipse.

3. The anatomical device of claim 1 wherein a portion of the flexible frame is arrangeable for applying local pressure to the dorsal vein of the penis and restricting the flow of blood out of the penis.

4. The anatomical device of claim 1 wherein the means for engaging the flexible frame in the locked position is a latex loop transversely secured over the distal ends of the left leg and right leg.

5. The anatomical device of claim 1 wherein the flexible frame is round.

6. The anatomical device of claim 1 wherein the flexible frame is rectangular.

7. The anatomical device of claim 1 wherein the left leg engaging section has a lower left engaging portion and the right engaging section has a lower right engaging portion, the lower left engaging portion and lower right engaging portion each configured to engage the underside of the penis.

8. The anatomical device of claim 7 wherein the lower left engaging portion and lower right engaging portion respectively engage the underside of the penis at an angle from 25 degrees to 35 degrees.

9. The anatomical device of claim 1 wherein the flexible frame is covered with a latex skin.

10. The anatomical device of claim 9 wherein two handles are formed of the latex skin, each handle comprising a loop of latex skin extending beyond each distal end and attaching to the latex skin covering the flexible frame.

11. The anatomical device of claim 10 wherein the means for engaging the flexible frame in the locked position is a latch adapted to lock the two handles together.

12. An anatomical device adapted for attachment to a penis, comprising:
(a) a frame for attachment to a penis, said frame having a left leg and a right leg, the left leg and right leg each having proximal and distal ends, the proximal end of the left leg attached to the proximal end of the right leg with pivoting attachment means, the left leg having an left engaging section, the right leg having a right engaging section, the left engaging section and right engaging section in spaced-apart facing relation, said left engaging section and right engaging section defining an elliptical opening for receiving the penis;
(b) said frame having a unlocked position, wherein the size of the elliptical opening defined by the left engaging section and right engaging section may manually be adjusted and a locked position wherein the size of said elliptical opening may not manually be adjusted; and
(c) means for engaging said frame in the locked position.

13. The anatomical device of claim 12 wherein the pivoting attachment means is a hinge.

14. The anatomical device of claim 12 wherein the elliptical opening defined by the left engaging section and right engaging section in the locked position is a 45 degree to 50 degree ellipse.

15. The anatomical device of claim 12 wherein the left leg engaging section has a lower left engaging portion and the right engaging section has a lower right engaging portion, the lower left engaging portion and lower right engaging portion each configured to engage the underside of the penis.

16. The anatomical device of claim 15 wherein the lower left engaging portion and lower right engaging portion respectively engage the underside of the penis at an angle from 25 degrees to 35 degrees.

17. The anatomical device of claim 12 wherein the means for engaging the frame in the locked position is a latex loop transversely secured over the distal ends of the left leg and right leg.

18. The anatomical device of claim 12 wherein the means for engaging the frame in the locked position is a rubber sleeve secured over the proximal ends of the left leg and right leg.

19. The anatomical device of claim 13 wherein the means for engaging the frame in the locked position is an adjustable fastener connecting the proximal end of the left leg to the proximal end of the right leg, said fastener mounted transversely to the hinge.

20. The anatomical device of claim 18 wherein the means for engaging the frame in the unlocked position is an adjustable fastener displacing the proximal end of the left leg from the proximal end of the right leg, said fastener mounted transversely to the hinge.

21. The anatomical device of claim 20 wherein a spring, having a left end and a right end, is attached to the frame, the left end being fastened to the proximal end of the left leg and the right end being fastened to the proximal end of the right leg.

22. An anatomical device adapted for attachment to a penis, comprising:
(a) a flexible frame for attachment to a penis, said flexible frame having a top, a left leg and a right leg, the left leg and right leg having distal ends, the left leg having an left engaging section, the right leg having a right engaging section, the left engaging section and right engaging section in spaced-apart facing relation, said top, left engaging section and right engaging section defining a triangular opening for receiving the penis;
(b) the left leg engaging section having a lower left engaging portion and the right engaging section having a lower right engaging portion, the lower left engaging portion and lower right engaging portion each configured to engage the underside of the penis;
(c) said flexible frame having a unlocked position, wherein the size of the triangular opening defined by the top, left engaging section and right engaging section may manually be adjusted and a locked position wherein the size of said triangular opening may not manually be adjusted; and
(d) means for engaging said flexible frame in the locked position.

23. The anatomical device of claim 22 wherein the lower left engaging portion and lower right engaging portion respectively engage the underside of the penis at an angle from 40 degrees to 45 degrees.

24. The anatomical device of claim 22 wherein the means for engaging the flexible frame in the locked position is a latex loop transversely secured over the distal ends of the left leg and right leg.

25. The anatomical device of claim 22 wherein the flexible frame is round.

26. The anatomical device of claim 22 wherein the flexible frame is rectangular.

27. The anatomical device of claim 22 wherein the flexible frame is covered with a latex skin.

28. The anatomical device of claim 27 wherein two handles are formed of the latex skin, each handle comprising a loop of latex skin extending beyond each distal end and attaching to the latex skin covering the flexible frame.

29. The anatomical device of claim 28 wherein the means for engaging the flexible frame in the locked position is a latch adapted to lock the two handles together.

30. The anatomical device of claim 22 wherein a portion of the flexible frame is arrangeable for applying local pressure to the dorsal vein of the penis and restricting the flow of blood out of the penis.

31. An anatomical device adapted for attachment to a penis, comprising:
(a) a frame for attachment to a penis, said frame having a left leg and a right leg, the left leg and right leg each having proximal and distal ends, the proximal end of the left leg attached to the proximal end of the right leg with pivoting attachment means, the left leg having an left engaging section, the right leg having a right engaging section, the left engaging section and right engaging section in spaced-apart facing relation, said left engaging section and right engaging section defining a triangular opening for receiving the penis;
(b) the left leg engaging section having a lower left engaging portion and the right engaging section having a lower right engaging portion, the lower left engaging portion and lower right engaging portion each configured to engage the underside of the penis;
(c) said frame having a unlocked position, wherein the size of the triangular opening defined by the left engaging section and right engaging section may manually be adjusted and a locked position wherein the size of said triangular opening may not manually be adjusted; and
(d) means for engaging said frame in the locked position.

32. The anatomical device of claim 31 wherein the pivoting attachment means is a hinge.

33. The anatomical device of claim 31 wherein the lower left engaging portion and lower right engaging portion respectively engage the underside of the penis at an angle from 40 degrees to 45 degrees.

34. The anatomical device of claim 31 wherein the means for engaging the frame in the locked position is a latex loop transversely secured over the distal ends of the left leg and right leg.

35. The anatomical device of claim 31 wherein the means for engaging the frame in the locked position is a rubber sleeve secured over the proximal ends of the left leg and right leg.

36. The anatomical device of claim 32 wherein the means for engaging the frame in the locked position is an adjustable fastener connecting the proximal end of the left leg to the proximal end of the right leg, said fastener mounted transversely to the hinge.

37. The anatomical device of claim 35 wherein the means for engaging the frame in the unlocked position is an adjustable fastener displacing the proximal end of the left leg from the proximal end of the right leg, said fastener mounted transversely to the hinge.

38. An anatomical device adapted for attachment to a penis, comprising:
(a) a frame for attachment to a penis, said frame having a top, a left leg and a right leg, the left leg and right leg having distal ends, the left leg having an left engaging section, the right leg having a right engaging section, the left engaging section and right engaging section in spaced-apart facing relation, said top, left engaging section and right engaging section defining an elliptical opening for receiving the penis;
(b) the left leg engaging section comprising an upper left engaging portion and a lower left engaging portion and the right engaging section comprising an upper right engaging portion and a lower right engaging portion, the upper left engaging portion connected to the lower left engaging portion with pivoting attachment means and the upper right engaging portion connected to the lower right engaging portion with pivoting attachment means, the lower left engaging portion and lower right engaging portion each configured to engage the underside of the penis;
(c) said frame having a unlocked position, wherein the size of the elliptical opening defined by the top, left engaging section and right engaging section may manually be adjusted and a locked position wherein the size of said elliptical opening may not manually be adjusted; and
(d) means for engaging said frame in the locked position.

39. The anatomical device of claim 38 wherein the pivoting attachment means is a hinge.

40. The anatomical device of claim 38 wherein the elliptical opening defined by the top, left engaging section and right engaging section in the locked position is a 45 degree to 50 degree ellipse.

41. The anatomical device of claim 38 wherein the lower left engaging portion and lower right engaging portion respectively engage the underside of the penis at an angle from 25 degrees to 35 degrees.

42. The anatomical device of claim 38 wherein the means for engaging the frame in the locked position is a latex loop transversely secured over the distal ends of the left leg and right leg.
